(19) United States
(12) Patent Application Publication
(10) Pub. No.: US 2014/0018867 A1
(43) Pub. Date: Jan. 16, 2014

(54) PRECAUTION AGAINST JAMMING ON OPEN BONE SCREWS

(71) Applicants: Stefan Freudiger, Bremgarten (CH); Rolf Diener, Winkel (CH)

(72) Inventors: Stefan Freudiger, Bremgarten (CH); Rolf Diener, Winkel (CH)

(21) Appl. No.: 13/982,663

(22) PCT Filed: Jan. 30, 2012
(86) PCT No.: PCT/CH2012/000025

§ 371 Date: Sep. 30, 2013

(30) Foreign Application Priority Data
Feb. 4, 2011 (CH) 214/11

(51) Int. Cl. A61B 17/86

(52) U.S. Cl. CPC A61B 17/8605
USPC 606/308

(57) ABSTRACT

In a bone screw (1) with an open receptacle for a connection element, in particular a connection rod, the risk of jamming is reduced by avoiding beveled flights at the lead-in of the thread pair, at least on the thread halves (2) of the receptacle or the attachment element (4, 6, 7, 9) which can be placed onto the head of the screw.

The present invention relates to a bone screw according to the preamble of Claim 1. In particular, it relates to the thread geometry for attachment elements of connection rods on bone screws with open receptacles.

The present invention discloses precautions, with the aid of which jamming of internal screws or external nuts to be placed on open bone screws can be prevented or at least the probability of the occurrence of jamming can be reduced. Open bone screws, in particular pedicle screws, are very advantageous in that, for example, vertebrae-connection rods can be inserted from the top. However, a disadvantage in this case consists of the fact that the thread for receiving an attachment element consists of two halves and this therefore increases the risk of jamming.

No previously known precautions against jamming in the case of open bone screws were found in the prior art, namely with the German keyword “Verkanten” or the English keywords “jam” or “cant”. A reason for this could lie in the fact that, in the past, connection rods tended to be offered made of metal for fusions and, these days, tend to be offered made of polymers for dynamic systems. As a result of their higher rigidity, metallic connection rods can be offered with smaller diameters, which is why the open thread halves can be thicker and, in terms of circumference, be provided with a larger threaded portion.

By contrast, connection rods made out of polymers have greater diameters, and so, for spatial reasons, the open thread halves have to be kept as small as possible, leading to smaller thread wrap angles and hence an increased risk of jamming.

It is accordingly an object of the invention described below to provide precautions with the aid of which jamming of an internal screw or external nut should be prevented when being placed on an open bone screw. Here, the internal screw or the external nut within the meaning of an attachment element has the object of attaching a connection rod, inserted through the open bone screw, in said bone screw.

Such a bone screw is specified in claim 1. The further claims specify preferred embodiments.

The solution to this object is distinguished by virtue of the fact that the leading-in flights do not have beveled segments which, as a result of their chamfer, could provoke jamming.

It follows that the subject matter of the invention is the special thread lead-in geometry defined in Patent Claim 1.

The present invention will be described in more detail below on the basis of preferred exemplary embodiments, with reference being made to the drawings.

In these drawings, schematically:

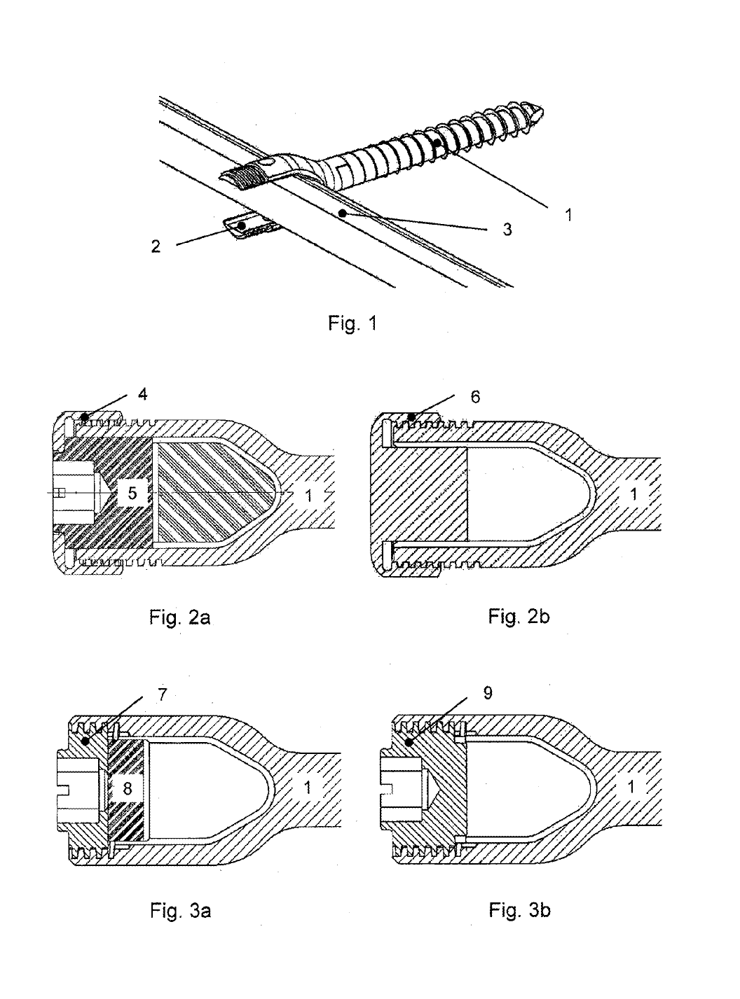

FIG. 1 shows a bone screw with an open receptacle for a connection rod.

FIG. 2a shows a rod attachment with external nut and filler piece.

FIG. 2b shows a rod attachment with an integral external nut and integrated filler piece.

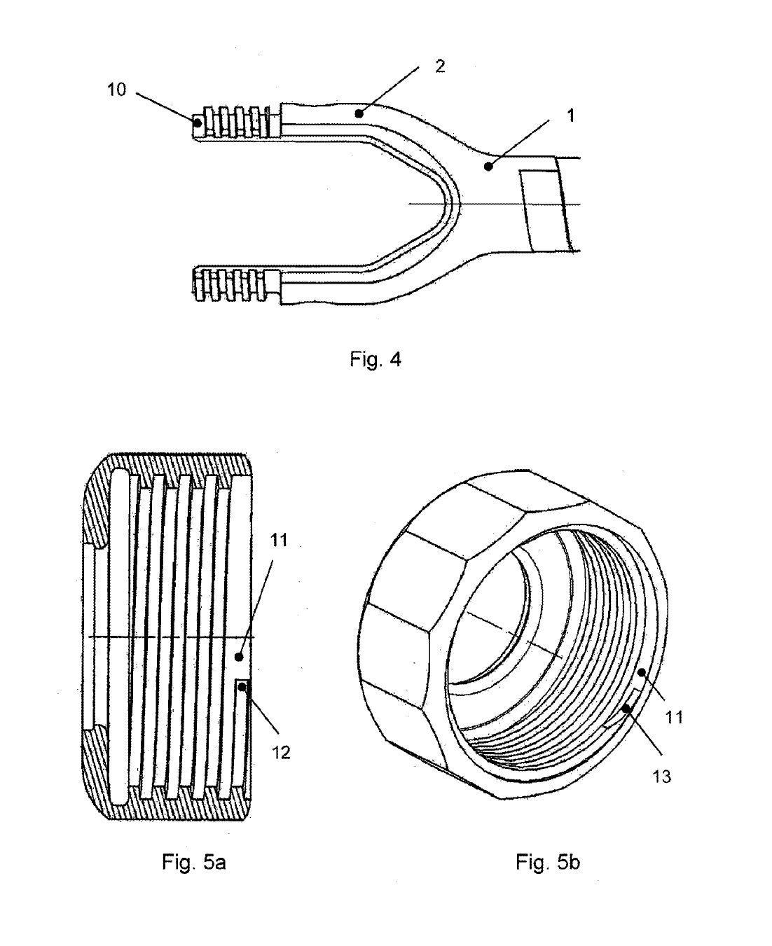

FIG. 3a shows a rod attachment with internal screw and filler piece.

FIG. 3b shows a rod attachment with integral internal screw and integrated filler piece.

FIG. 4 shows a bone screw with only whole flights on the outer side of the rod receptacle (male piece on the bone screw).

FIG. 5a shows an external screw, in a section, with only whole flights on the inner side (female piece in the attachment element).

FIG. 5b shows an internal view of the external nut with a rounded transition to the first flight at full height.

FIG. 1 shows a bone screw (1) with an open receptacle consisting of respectively one thread half (2) for receiving a connection rod (3).

The wrap angle, i.e. the portion of the circumference of the head of a bone screw which is formed by a thread half, is generally at most 150° and often at most 120°. Particularly in the case of receptacles for a polymer connection rod, angles of 90° and less preferably occur due to the larger opening relative to the head cross section.

FIG. 2a shows a bone screw (1) with a rod attachment consisting of an external nut (4) and filler piece (5).

FIG. 2b shows a bone screw (1) with a rod attachment consisting of an external nut with integrated filler piece (6).

FIG. 3a shows a bone screw (1) with a rod attachment consisting of an internal screw (7) and filler piece (8).

FIG. 3b shows a bone screw (1) with a rod attachment consisting of an internal screw and integrated filler piece (9).

FIG. 4 shows a bone screw (1) with respectively one thread half (2) with only whole flights (10) on the external side of the rod receptacle (male piece on the bone screw).

FIG. 5a shows a section of an external nut with only whole flights (11) (female piece in the attachment element), in which the lead-in of the first thread pitch with full height is oblique (12).

FIG. 5b shows an internal view of an external nut with no beveled flights (11) (female piece in the attachment element), in which the lead-in of the first thread pitch with full height is slightly rounded (13).

An oblique (12) or only slightly rounded (13) thread lead-in with a thread flank at full height is additionally advantageous in that, if it cannot ensure correct engagement immediately, it leads to a fast and clearly perceivable abutment. In such a case, it suffices for the surgeon to turn the attachment element back by a little more than 180°, which then brings him automatically to the correct relative position of the threads to be brought together.

Of course, this invention is not restricted to single flights, but can accordingly also be applied to multiple flights.

The precaution against jamming can be further supported by virtue of a filler piece (5, 8) being provided with tight tolerances and contributing to the correct centering of the thread pairs if said filler piece is used. The precaution against jamming can be further supported by virtue of the correctly fitting thread halves each being marked by a different color or different shading on the male and female side. For the attachment element, the drawings show in detail a male thread on the external side of the bone screw thread halves and a female thread on the inner side of the attachment element. This invention can naturally also be applied the other way around, namely to a male thread on the external side of the attachment element and a female thread on the inner side of the bone screw thread halves, which are not drawn here.

The preceding description of preferred exemplary embodiments does not restrict the scope of protection of the present invention, which is defined solely by the claims, and enables a person skilled in the art to undertake modifications and additions within the scope of protection.

1. Bone screw with two thread halves, which delimit an open receptacle for a connection element, and attachment element, wherein the thread halves are designed for receiving the attachment element, wherein at least one attachment member selected from the thread halves and the attachment element has a thread lead-in, in which flights are only present when these are formed at a full flank height.

2. Bone screw and attachment element according to claim 1, wherein the thread lead-in of the at least one attachment member does not have any beveled flights.

3. Bone screw and attachment element according to claim 1, wherein the attachment element is an external nut and a thread for receiving the latter is situated on the external side of the bone screw thread halves.

4. Bone screw and attachment element according to claim 1, wherein the attachment element is an internal screw and a thread for receiving the latter is situated on the inner side of the bone screw thread halves.

5. Bone screw and attachment element according to claim 3, wherein the thread has one or more flights.

6. Bone screw and attachment element according to claim 5, wherein the attachment element is combined with a filler piece, which additionally serves as centering aid.

7. Bone screw and attachment element according to claim 5, wherein additional jamming protection is applied in the form of a different color marking or shading per thread half and associated half of the attachment element.

8. Bone screw and attachment element according to claim 1, wherein flights are only present on the attachment element and the thread halves when these are formed at the full flank height.

9. Bone screw and attachment element according to claim 7, wherein the attachment element and the thread halves do not have any beveled flights.
